(19) United States
(12) Patent Application Publication
(10) Pub. No.: US 2025/0336525 A1
(43) Pub. Date: Oct. 30, 2025

(54) METHOD AND SYSTEM FOR PREDICTING EFFICACY OF NEOADJUVANT CHEMOTHERAPY FOR LOCALLY ADVANCED GASTRIC CANCER

(71) Applicant: Cancer Hospital of China Academy of Medical Sciences, Beijing (CN)

(72) Inventors: Yongjian ZHU, Beijing (CN); Liming JIANG, Beijing (CN); Xinming ZHAO, Beijing (CN)

(73) Assignee: Cancer Hospital of China Academy of Medical Sciences, Beijing (CN)

(21) Appl. No.: 19/002,799

(22) Filed: Dec. 27, 2024

(30) Foreign Application Priority Data
Apr. 28, 2024 (CN) 202410520272.8

(51) Int. Cl. G16H 50/20; G06V 10/25; G06V 10/26; G06V 10/28; G06V 10/72; G06V 10/774; G06V 10/82; G16H 10/60; G16H 20/10; G16H 30/40

(52) U.S. Cl. CPC G16H 50/20; G06V 10/25; G06V 10/267; G06V 10/28; G06V 10/72; G06V 10/774; G06V 10/82; G16H 10/60; G16H 20/10; G16H 30/40

(57) ABSTRACT

A method and a system for predicting the efficacy of neoadjuvant chemotherapy for locally advanced gastric cancer are provided. The method includes the following steps: the historical image data acquired through multi-b-values non-Gaussian diffusion MRI and corresponding prognostic image data are acquired, so that to obtain the signal intensity data corresponding to different b values, and influencing factors are acquired by combining diffusion models; a tumor tissue image is selected the region of interest which is further performed data labeling and enhancement to obtain a label set; the deep perception network is established to divide efficacy levels; A data set is established according to the influencing factors and the efficacy levels, a CNN-LSTM prediction model is constructed, the CNN-LSTM prediction model is optimized by using the data set to obtain an optimal model, and the chemotherapy efficacy level is evaluated by the optimal model.

### CROSS REFERENCE TO THE RELATED APPLICATIONS

This application is based upon and claims priority to Chinese Patent Application No. 202410520272.8, filed on Apr. 28, 2024, the entire contents of which are incorporated herein by reference.

### TECHNICAL FIELD

The present disclosure relates to the technical field of biomedicine, more specifically to a method and a system for predicting the efficacy of neoadjuvant chemotherapy for locally advanced gastric cancer.

### BACKGROUND

In recent years, the neoadjuvant chemotherapy (NCT) has been proved to significantly improve the prognosis of patients with locally advanced gastric cancer (LAGC), and has become a standard treatment. More and more evidence showed that NCT could induce tumor downstaging, reduce tumor volume, increase surgical resectability and R0 resection rate, eliminate micrometastasis to reduce recurrence and improve patient survival, but the overall effectiveness rate of patients to NCT is still less than 50%, and the prognosis is not ideal. However, not all LAGC patients can benefit from NCT, and ineffective neoadjuvant therapy may increase chemotherapy toxicity and lead to tumor progression during treatment, delaying the timing of surgery. Therefore, the current research is constantly exploring how to predict the benefit results of patients in the early stage of preoperative chemotherapy or before treatment.

The research developed machine learning models (radiomics) to noninvasively identify patients who would respond to chemotherapy based on clinicopathological data and CT images. Pretherapeutic imaging is related to the features of primary tumors, and postoperative imaging can reflect the response and effect of the tumor treatment. The research found that the internal heterogeneity of the tumor can be reflected to a certain extent by the unique texture and spatial gray pattern of the extracted radiomics features in the CT image. Therefore, the current research focuses on development of machine learning models (radiomics) using CT images to noninvasively identify patients who would respond to chemotherapy, including: the extraction and selection of radiomics features, the establishment and verification of the multimodal machine learning models, etc.

However, there are still many omissions and misdetections in the verification process of the current prediction model of the neoadjuvant chemotherapy response in gastric cancer based on the radiomics, and the stability of the radiomics model needs to be further verified. The bio-interpretability of its features is poor, and the image features are artificially set, resulting in the failure to fully capture the features of high sensitivity to a certain disease, so that the actual prediction is biased.

Based on different fitting algorithms, non-Gaussian diffusion-weighted imaging with multi-b-values (By collecting DWI images with multiple consecutive b-values) can obtain multiple quantitative parameters, such as intravoxel incoherent motion model and the diffusion kurtosis model, respectively, reflecting the cell proliferation activity, blood perfusion and tumor heterogeneity of the tumor tissue. The imaging technology has the advantages of non-invasive, in vivo detection and high repeatability and the like.

Therefore, it is an urgent problem for those skilled in the art to better predict the efficacy of the chemotherapy prognosis. The non-Gaussian diffusion-weighted imaging with multi-b-values provides a powerful tool for predicting the efficacy of the neoadjuvant chemotherapy for locally advanced gastric cancer.

### SUMMARY

In view of this, the present disclosure provides a method and a system for predicting the efficacy of the neoadjuvant chemotherapy for locally advanced gastric cancer, which enhance the precision of the efficacy prediction of the neoadjuvant chemotherapy for locally advanced gastric cancer by calculating the parameters of the intravoxel incoherent motion model and the diffusion kurtosis model combined with the convolutional neural network and combining different diffusion models, which can more accurately fit the complex mode of efficacy data.

In order to achieve the above purpose, the present disclosure adopts the following technical solutions:

A method for predicting the efficacy of the neoadjuvant chemotherapy for locally advanced gastric cancer includes:
acquiring the historical image data of local progression acquired through multi-b-values non-Gaussian diffusion magnetic resonance imaging (MRI) and corresponding prognostic image data acquired periodically, and dividing the image data according to the preset b-value sequence to obtain the signal intensity data corresponding to each b-value;
acquiring the voxel-related parameter and the diffusion-related parameter according to the signal intensity data combined with the intravoxel incoherent motion model and the diffusion kurtosis model; taking the voxel-related parameter and the diffusion-related parameter as influencing factors;
selecting the optimal gray threshold for the segmentation of the prognostic image data, extracting the region of interest according to the optimal gray threshold; performing the gray segmentation and the gradient segmentation on the region of interest respectively, and further adopting the two-peak iterative algorithm for binary segmentation to obtain the gray binary image and the gradient binary image; merging to form the segmentation candidate region, dividing the segmentation candidate region into blocks, and further performing data labeling and enhancement to obtain a label set;
establishing the deep perception network, taking the prognostic image data as the training set; inputting the label set and the training set into the deep perception network to obtain the tumor region and the fibrosis region to divide efficacy levels; and
establishing the data set by preprocessing the influencing factors and the efficacy levels; at the same time, constructing the convolutional neural network (CNN)-long short-term memory (LSTM) prediction model, and setting the initial parameter value of the model; adopting the data set to perform hyperparameter optimization to CNN-LSTM prediction model to obtain the optimal model, and evaluating the chemotherapy efficacy level of the predicted image by the optimal model.

Preferably, the preset b-value sequence includes 0, 10, 20, 50, 100, 200, 400, 600, 800, 1000, 1500, 2000 specifically; the unit is s/mm².

Preferably, the voxel-related parameter includes a slow diffusion coefficient, a fast diffusion coefficient and a perfusion fraction; the diffusion-related parameter includes an average diffusion rate and average diffusion kurtosis.

Preferably, the optimal gray threshold specifically includes:

$${k}^{*} = {arg}_{k} ( {max}_{1 \leq k \leq L} {σ}_{B}^{2} ( k ) ) ; \qquad [ 19 ]$$

wherein, L is the level of the gray histogram; k is the gray threshold to distinguish the regional target from the targets that are significantly different from the regional targets;

$${σ}_{B}^{2}$$

is the overall inter-class difference of MRI images,

$${σ}_{B}^{2} = {w}_{1} {{w}_{2} ( {μ}_{1} - {μ}_{2} )}^{2} ,$$

w₁ is the appearing probability of the regional targets, w₂ is the appearing probability of the targets that are significantly different from the regional targets, μ₁ is the average gray of the regional targets, and μ₂ is the average gray of the targets that are significantly different from the regional targets.

Preferably, the performing data labeling and enhancement includes:
labeling the training data: labeling tissue according to whether it is a tumor tissue or not, and setting a discriminant threshold A here; if the proportion of the fibrosis in the gray binary image and the gradient binary image of the block region after block processing exceeds A, the block region is considered to be a tumor tissue, and the label is set to 1; otherwise, the label is fibrosis tissue, and set to be 0; and
enhancing the training data: the data enhancement includes rotation, mirror image, and gray transformation: the rotation is to rotate the block region according to a certain angle step;
the mirror image is to turn the block region horizontally and vertically to obtain the image after the mirror operation; the gray transformation is to compress and stretch the gray of the block region to obtain a plurality of groups of transformed pictures as a label set, and the transformation scale factor is δ.

Preferably, the establishing the deep perception network includes:
S1: constructing an exponential linear unit (ELU) activation function to alleviate the disappearance of linear gradient;
S2: constructing a deep separable convolution module further by using the ELU activation function: the module includes a 3×3 convolution and a 1×1 convolution, and the number of the convolution kernels is 64 and 128 respectively; and
S3: constructing a residual module by using the deep separable convolution module: the combination of the residual module structure is as follows: it is includes two branches, the uplink branch includes a mean pooling layer, and the downlink branch includes the 3×3 convolution layer→the ELU activation layer→the deep separable convolution layer→the ELU activation layer→the 1×1 convolution layer→the ELU activation layer, wherein, the convolution kernel dimensions of the 3×3 convolution layer and the 1×1 convolution layer are 64 and 128 respectively. Finally, the uplink branch and the downlink branch are connected by the Contact layer.

Preferably, the specific structure of the deep perception network is: the data input layer→the convolution layer 1→the convolution layer 2→the residual module→the residual module→the residual module→the convolution layer 3→the global mean pooling→the softmax layer; wherein the convolution layer 1, the convolution layer 2 and the convolution layer 3 are all 3×3 convolution kernels, and the kernel dimensions are 16, 32 and 128 respectively. The softmax layer outputs the final image block probability attribute, that is, the category corresponding to the high probability value is the category of the image block, and the calculation formula is:

$${y}_{i} = {S ( z )}_{i} = \frac{{e}^{{z}_{i}}}{{\sum }_{j = 1}^{C} {e}^{{z}_{j}}} , i = 1 , ... , C ; \qquad [ 29 ]$$

wherein z is the output of the previous layer, the input dimension of softmax is C, and yᵢ is the probability that the predicted object belongs to class C.

Preferably, the constructing of CNN-LSTM prediction model includes: the input layer, the hidden layer and the output layer; firstly, the data is organized into a form that the prediction model can recognize and imported the data into the prediction model through the input layer; then the data is processed through the hidden layer of the core part, in which the CNN layer analyzes the correlation features between the efficacy level and its influencing factors, and then the LSTM layer extracts the features of the time series data in the time dimension; then, the complexity of the model is increased through the Dense layer, and the data is mapped from high dimension to low dimension to retain useful information. At the same time, a Dropout layer is connected after each layer to enhance the robustness of the model and prevent the model from overfitting. Finally, the predicted value is output through the output layer. The input of the model is a two-dimensional matrix including the efficacy level vₜ [symbol] vₜ₋₁ [symbol] . . . [symbol] v_{t-λ+1} and the influencing factor

$${f}_{t}^{( i )} , {f}_{t - 1}^{( i )} , ... , {f}_{t - λ + 1}^{( i )} , ( i = 1 , 2 , 3 , ... k ) ,$$

and the size of the two-dimensional matrix is (λ, k+1).

A system for predicting the efficacy of the neoadjuvant chemotherapy for locally advanced gastric cancer includes:
a data acquisition module, configured to acquire the historical image data of local progression acquired through multi-b-values non-Gaussian diffusion MRI and corresponding prognostic image data acquired periodically, and divide the image data according to the preset b-value sequence to obtain the signal intensity data corresponding to each b-value;
a factor acquisition module, configured to acquire the voxel-related parameter and the diffusion-related parameter according to the signal intensity data combined with the intravoxel incoherent motion model and the diffusion kurtosis model, and take the voxel-related parameter and the diffusion-related parameter as influencing factors;
a label set establishment module, configured to select the optimal gray threshold for the segmentation of the prognostic image data, extract the interest region according to the optimal gray threshold; perform the gray segmentation and the gradient segmentation on the interest region, and further adopt the two-peak iterative algorithm for binary segmentation to obtain the gray binary image and the gradient binary image; merge to form the segmentation candidate region, divide the segmentation candidate region into blocks, and further perform the data labeling and the enhancement to obtain a label set;
a level division module, configured to establish the deep perception network, take the prognostic image data as the training set; input the label set and the training set into the deep perception network to obtain the tumor region and the fibrosis region to divide efficacy levels; and
a level prediction module, configured to establish the data set by preprocessing the influencing factors and the efficacy levels; at the same time, construct the CNN-LSTM prediction model, and set the initial parameter value of the model; adopt the data set to perform Hyperparameter optimization to CNN-LSTM prediction model to obtain the optimal model, and evaluate the chemotherapy efficacy level of the predicted image by the optimal model.

Through the above technical solutions, compared with the prior art, the method and the system for predicting the efficacy of neoadjuvant chemotherapy for locally advanced gastric cancer provided by the present disclosure have the following beneficial effects:
(1) the efficacy level of the tumor chemotherapy can be predicted more accurately, which can provide a more reliable diagnostic basis for clinicians by combining the voxel-related parameter, the diffusion-related parameter, and the deep perception network technology;
(2) the automatic processing and analysis of a large number of MRI image data can be realized, and the work efficiency and accuracy can be improved by using the deep perception network and the CNN-LSTM prediction model;
(3) the key information of the region of interest can be effectively extracted to help clinicians better understand the lesions by data processing and image segmentation technology; and
(4) it has a driving effect on research in the fields of medical image processing, deep learning, and medical prediction, which helps to promote scientific research progress in related fields by combining multiple advanced technologies.

### BRIEF DESCRIPTION OF THE DRAWINGS

In order to more clearly illustrate the embodiments of the present disclosure or technical solutions in the related art, the accompanying drawings used in the embodiments or the related art will now be described briefly. It is obvious that the drawings in the following description are only the embodiment of the disclosure, and that those skilled in the art can obtain other drawings from these drawings without any creative efforts.

FIG. 1 is a flow chart of the method steps provided by the present disclosure; and

FIG. 2 is a flow chart of the deep perception network establishment provided by the present disclosure.

### DETAILED DESCRIPTION OF THE EMBODIMENTS

In the following, the technical solutions in the embodiments of the present disclosure will be clearly and completely described with reference to the drawings in the embodiments of the present disclosure. Obviously, the described embodiments are only a part of the embodiments of the present disclosure, but not all the embodiments thereof. Based on the embodiments of the present disclosure, all other embodiments obtained by those skilled in the art without any creative efforts shall fall within the scope of the present disclosure.

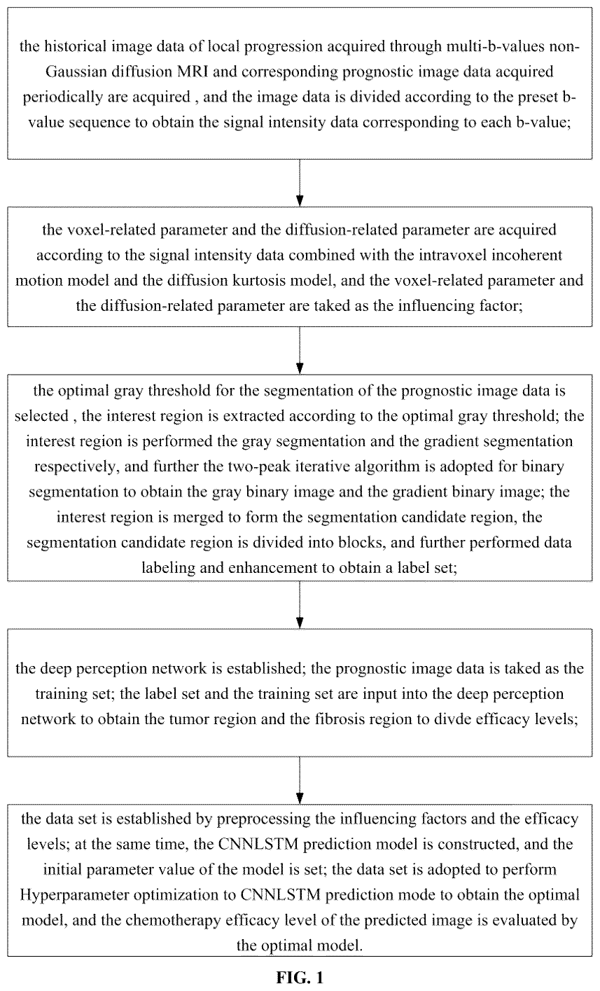

As shown in FIG. 1, The embodiment of the present disclosure discloses a method for predicting the efficacy of the neoadjuvant chemotherapy for locally advanced gastric cancer, including:
the historical image data of local progression acquired through multi-b-values non-Gaussian diffusion MRI and corresponding prognostic image data acquired periodically are acquired, and the image data is divided according to the preset b-value sequence to obtain the signal intensity data corresponding to each b-value;
the voxel-related parameter and the diffusion-related parameter are acquired according to the signal intensity data combined with the intravoxel incoherent motion model and the diffusion kurtosis model, and the voxel-related parameter and the diffusion-related parameter are taken as the influencing factor;
the optimal gray threshold for the segmentation of the prognostic image data is selected, the region of interest is extracted according to the optimal gray threshold; the region of interest is performed the gray segmentation and the gradient segmentation respectively, and further the two-peak iterative algorithm is adopted for binary segmentation to obtain the gray binary image and the gradient binary image; the interest region is merged to form the segmentation candidate region, the segmentation candidate region is divided into blocks, and further performed data labeling and enhancement to obtain a label set;
the deep perception network is established; the prognostic image data is taken as the training set; the label set and the training set are input into the deep perception network to obtain the tumor region and the fibrosis region to divide efficacy levels; and
the data set is established by preprocessing the influencing factors and the efficacy levels; at the same time, the CNN-LSTM prediction model is constructed, and the initial parameter value of the model is set; the data set is adopted to perform hyperparameter optimization to CNN-LSTM prediction model to obtain the optimal model, and the chemotherapy efficacy level of the predicted image is evaluated by the optimal model.

In a specific embodiment, the method for performing the hyperparameter optimization on the prediction model through the data set includes the following steps:
Step 1: the size of the convolution kernel and the pooling mode are determined;
Step 2: the weights and the biases of the CNN-LSTM prediction model are initialized;
Step 3: the data set data is imported into the CNN-LSTM prediction model, and the model output result is calculated by using the forward propagation algorithm;
Step 4: the error between the model output result and the real value is calculated;
Step 5: the gradient is calculated by using the back propagation algorithm according to the error;
Step 6, the parameters are updated according to the gradient size; and
Step 7, the step 3 to the step 6 are repeated until the training condition is satisfied and outputting the optimal model.

In a specific embodiment, the tumor response was divided into five levels by the Mandalay tumor regression level system to classify treatment efficacy levels:
TRG 1 (complete tumor regression);
TRG 2 (scattered tumor cells in fibrosis);
TRG 3 (tumor cells and fibrosis, mainly fibrosis);
TRG 4 (tumor cells and fibrosis, mainly tumor cells);
TRG 5 (no tumor regression).

The patients were divided into two groups: pathological responders (TRG 1-3) and pathological non-responders (TRG 4-5).

In a specific embodiment, the non-Gaussian diffusion MRI sequence was used, and the respiratory trigger mode STIR-EPI DWI acquisition was adopted. The preset b-value sequence included 0, 10, 20, 50, 100, 200, 400, 600, 800, 1000, 1500, 2000; the unit is s/mm². The number of excitation (NEX) were 1 (the b-value were 0, 10, 20, 50, 100, 200, 400 s/mm²), 2 (the b-value were 600,800 s/mm²), 3 (the b-value were 1000, 1200, 1500 s/mm²) and 4 (the b-value is 2000 s/mm²).

In a specific embodiment, the intravoxel incoherent motion model is calculated by using 11 b-values (0, 10, 20, 50, 100, 200, 400, 600, 800, 1000 s/mm²), including:

$$\frac{{S}_{b}}{{S}_{0}} = {f}^{- b \cdot ( D + {D}^{*} )} + {( 1 - f )}^{- b \cdot D} ; \qquad [ 70 ]$$

wherein, b is the diffusion gradient factor, S_{b} is the signal intensity if the gradient magnetic field intensity in diffusion imaging is b, S₀ is the signal intensity if the gradient magnetic field intensity in diffusion imaging is 0, D is the slow diffusion coefficient, D* is the perfusion-related diffusion coefficient or the fast diffusion coefficient, and f is the perfusion fraction.

The diffusion kurtosis model is calculated by using four b-values (0, 800, 1500, 2000 s/mm²), including:

$$\frac{{S}_{b}}{{S}_{0}} = exp ( - b \cdot M D + \frac{1}{6} \cdot {b}^{2} \cdot M {D}^{2} \cdot M K ) \qquad [ 73 ]$$

wherein, MD is the average diffusivity and MK is the average diffusion kurtosis.

In a specific embodiment, the voxel-related parameter includes the slow diffusion coefficient, the fast diffusion coefficient and the perfusion fraction. The diffusion-related parameter includes the average diffusion rate and the average diffusion kurtosis.

In a specific embodiment, the optimal grayscale threshold specifically includes:

$${k}^{*} = {arg}_{k} ( {max}_{1 \leq k \leq L} {σ}_{B}^{2} ( k ) ) ; \qquad [ 77 ]$$

wherein, L is the level of the gray histogram; k is the gray threshold to distinguish the regional target from the targets that are significantly different from the regional targets;

$${σ}_{B}^{2}$$

is the overall inter-class difference of MRI images,

$${σ}_{B}^{2} = {w}_{1} {{w}_{2} ( {μ}_{1} - {μ}_{2} )}^{2} ,$$

w₁ is the appearing probability of the regional targets, w₂ is the appearing probability of the targets that are significantly different from the regional targets, μ₁ is the average gray of the regional targets, and μ₂ is the average gray of the targets that are significantly different from the regional targets.

In a specific embodiment, preform binary segmentation by utilizing the two-peak iterative algorithm includes the following steps: firstly, the histogram statistics is performed on the image, and the histogram is divided into two intervals by giving the initial iterative threshold; then, the gray or gradient mean of each interval pixel is calculated respectively; secondly, the average value of the gray or gradient mean of each interval is used as the next iteration threshold for repeated iteration; finally, stop the iteration until the threshold of the two iterations is less than the fixed threshold, and the image is binarized with the current threshold as the segmentation threshold.

In a specific embodiment, the data labeling and the enhancement include:
labeling the training data: the tissue is labelled according to whether it is a tumor tissue or not, and a discriminant threshold A is set here; if the proportion of the fibrosis in the gray binary image and the gradient binary image of the block region after block processing exceeds A, the block region is considered to be a tumor tissue, and the label is set to 1; otherwise, the label is fibrosis tissue, and set to be 0; and
enhancing the training data: the data enhancement includes rotation, mirror image, and gray transformation: the rotation is to rotate the block region according to a certain angle step; the mirror image is to turn the block region horizontally and vertically to obtain the image after the mirror operation; the gray transformation is to compress and stretch the gray of the block region to obtain a plurality of groups of transformed pictures as a label set, and the transformation scale factor is δ.

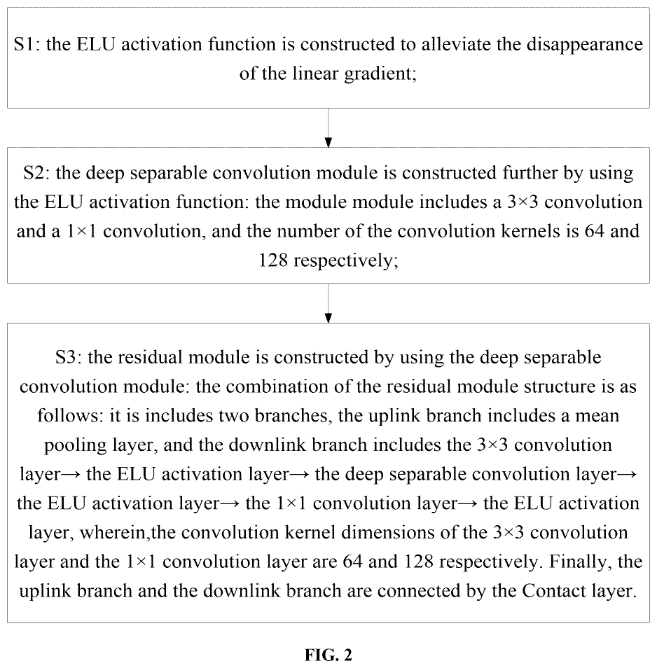

In a specific embodiment, the establishing the deep perception network is as shown in FIG. 2, which specifically includes:
S1: the ELU activation function is constructed to alleviate the disappearance of the linear gradient;
S2: the deep separable convolution module is constructed further by using the ELU activation function: the module includes a 3×3 convolution and a 1×1 convolution, and the number of the convolution kernels is 64 and 128 respectively; and
S3: the residual module is constructed by using the deep separable convolution module: the combination of the residual module structure is as follows: it is includes two branches, the uplink branch includes a mean pooling layer, and the downlink branch includes the 3×3 convolution layer→the ELU activation layer→the deep separable convolution layer→the ELU activation layer→the 1×1 convolution layer→the ELU activation layer, wherein, the convolution kernel dimensions of the 3×3 convolution layer and the 1×1 convolution layer are 64 and 128 respectively. Finally, the uplink branch and the downlink branch are connected by the Contact layer.

In a specific embodiment, the ELU activation function is

$$f ( x ) = \{ \begin{matrix}x & if x \geq 0 \\ α ( {e}^{x} - 1 ) & if x < 0\end{matrix} ,$$

wherein, x is the input and α is the regulator.

In a specific embodiment, the specific structure of the deep perception network is: the data input layer→the convolution layer 1→the convolution layer 2→the residual module→the residual module→the residual module→the convolution layer 3→the global mean pooling→the softmax layer; wherein the convolution layer 1, the convolution layer 2 and the convolution layer 3 are all 3×3 convolution kernels, and the kernel dimensions are 16, 32 and 128 respectively. The softmax layer outputs the final image block probability attribute, that is, the category corresponding to the high probability value is the category of the image block, and the calculation formula is:

$${y}_{i} = {S ( z )}_{i} = \frac{{e}^{{z}_{i}}}{{\sum }_{j = 1}^{C} {e}^{{z}_{j}}} , i = 1 , … , C ;$$

wherein z is the output of the previous layer, the input dimension of softmax is C, and yᵢ is the probability that the predicted object belongs to class C.

In a specific embodiment, the constructing of CNN-LSTM prediction model includes: the input layer, the hidden layer and the output layer; firstly, the data is organized into a form that the prediction model can recognize and imported the data into the prediction model through the input layer; then the data is processed through the hidden layer of the core part, in which the CNN layer analyzes the correlation features between the efficacy level and its influencing factors, and then the LSTM layer extracts the features of the time series data in the time dimension; then, the complexity of the model is increased through the Dense layer, and the data is mapped from high dimension to low dimension to retain useful information. At the same time, a Dropout layer is connected after each layer to enhance the robustness of the model and prevent the model from overfitting. Finally, the predicted value is output through the output layer. The input of the model is a two-dimensional matrix including the efficacy level vₜ [symbol] vₜ₋₁ [symbol] . . . [symbol] v_{t-λ+1} and the influencing factor

$${f}_{t}^{( i )} , {f}_{t - 1}^{( i )} , … , {f}_{t - λ + 1}^{( i )} , ( i = 1 , 2 , 3 , … k ) ,$$

and the size of the two-dimensional matrix is (λ, k+1).

In the initialization of model parameters, the size of the convolution kernel is set to 3*3 and average pooling is adopted. The Adam algorithm is adopted as the optimization algorithm of the model, the MSE is adopted as the loss function, the ReLU is adopted as the activation function, and the dropout method is adopted to prevent the model from overfitting. In addition, there are hyperparameters such as the step size, the batch size, the number of the iterations, the number of the filters, the number of the neurons in the LSTM layer, and the sensitivity analysis is performed on each hyperparameter. In the sensitivity analysis of a certain hyperparameter, the values of other hyperparameters are fixed.

The system for predicting the efficacy of the neoadjuvant chemotherapy for locally advanced gastric cancer includes:
a data acquisition module, which is configured to acquire the historical image data of local progression acquired through multi-b-values non-Gaussian diffusion MRI and corresponding prognostic image data acquired periodically, and divide the image data according to the preset b-value sequence to obtain the signal intensity data corresponding to each b-value;
a factor acquisition module, which is configured to acquire the voxel-related parameter and the diffusion-related parameter according to the signal intensity data combined with the intravoxel incoherent motion model and the diffusion kurtosis model, and take the voxel-related parameter and the diffusion-related parameter as influencing factors;
a label set establishment module, which is configured to select the optimal gray threshold for the segmentation of the prognostic image data, extract the region of interest according to the optimal gray threshold; perform the gray segmentation and the gradient segmentation on the region of interest, and further adopt the two-peak iterative algorithm for binary segmentation to obtain the gray binary image and the gradient binary image; the region of interest is merged to form the segmentation candidate region, divide the segmentation candidate region into blocks, and further perform the data labeling and the enhancement to obtain a label set;
a level division module, which is configured to establish the deep perception network, take the prognostic image data as the training set; input the label set and the training set into the deep perception network to obtain the tumor region and the fibrosis region to divide efficacy levels; and
a level prediction module, which is configured to establish the data set by preprocessing the influencing factors and the efficacy levels; at the same time, construct the CNN-LSTM prediction model, and set the initial parameter value of the model; adopt the data set to perform Hyperparameter optimization to CNN-LSTM prediction model to obtain the optimal model, and evaluate the chemotherapy efficacy level of the predicted image by the optimal model.

Various embodiments of the present specification are described in a progressive manner, and each embodiment focuses on the description that is different from the other embodiments, and the same or similar parts between the various embodiments are referred to with each other. For the device disclosed in the embodiment, since it corresponds to the method disclosed in the embodiment, the description is relatively simple, and the correlation is described with reference to the method part.

The above description of the disclosed embodiments enables those skilled in the art to implement or use the present disclosure. Various amendments to the embodiments will be apparent to those skilled in the art. The general principles defined herein may be implemented in other embodiments without departing from the spirit or scope of the disclosure. Therefore, the present disclosure will not be limited to the embodiments shown herein but is to be accorded the widest scope consistent with the principles and novel features disclosed herein.

What is claimed is:

1. A method for predicting an efficacy of neoadjuvant chemotherapy for locally advanced gastric cancer, comprising:
acquiring historical image data of a local progression acquired through multi-b-values non-Gaussian diffusion magnetic resonance imaging (MRI) and corresponding prognostic image data acquired periodically, and dividing the historical image data according to a preset b-value sequence to obtain signal intensity data corresponding to each b-value;
acquiring a voxel-related parameter and a diffusion-related parameter according to the signal intensity data combined with an intravoxel incoherent motion model and a diffusion kurtosis model; taking the voxel-related parameter and the diffusion-related parameter as influencing factors;
selecting an optimal gray threshold for segmentation of the prognostic image data, extracting an interest region according to the optimal gray threshold; performing gray segmentation and gradient segmentation on the interest region respectively, and further adopting a two-peak iterative algorithm for binary segmentation to obtain a gray binary image and a gradient binary image; merging to form a segmentation candidate region, dividing the segmentation candidate region into blocks, and further performing data labeling and data enhancement to obtain a label set;
establishing a deep perception network, taking the prognostic image data as a training set; inputting the label set and the training set into the deep perception network to obtain a tumor region and a fibrosis region to divide efficacy levels; and
establishing a data set by preprocessing the influencing factors and the efficacy levels; constructing a convolutional neural network (CNN)-long short-term memory (LSTM) prediction model, and setting an initial parameter value of the CNN-LSTM prediction model simultaneously; adopting the data set to perform Hyperparameter optimization to the CNN-LSTM prediction model to obtain an optimal model, and evaluating a chemotherapy efficacy level of a predicted image by the optimal model.

2. The method for predicting the efficacy of the neoadjuvant chemotherapy for the locally advanced gastric cancer according to claim 1, wherein the preset b-value sequence comprises 0, 10, 20, 50, 100, 200, 400, 600, 800, 1000, 1500, and 2000; a unit is s/mm².

3. The method for predicting the efficacy of the neoadjuvant chemotherapy for the locally advanced gastric cancer according to claim 1, wherein the voxel-related parameter comprises a slow diffusion coefficient, a fast diffusion coefficient, and a perfusion fraction; the diffusion-related parameter comprises an average diffusion rate and average diffusion kurtosis.

4. The method for predicting the efficacy of the neoadjuvant chemotherapy for the locally advanced gastric cancer according to claim 1, wherein the optimal gray threshold comprises:
$${k}^{*} = {arg}_{k} ( {max}_{1 \leq k \leq L} {σ}_{B}^{2} ( k ) ) ;$$
wherein L is a level of a gray histogram; k is a gray threshold to distinguish a regional target from targets, the targets are significantly different from the regional targets;
$${σ}_{B}^{2}$$
is an overall inter-class difference of MRI images,
$${σ}_{B}^{2} = {w}_{1} {{w}_{2} ( {μ}_{1} - {μ}_{2} )}^{2} ,$$
w₁ is an appearing probability inter-class difference of MRI images, of the regional targets, w₂ is an appearing probability of the targets, μ₁ is an average gray of the regional targets, and μ₂ is an average gray of the targets.

5. The method for predicting the efficacy of the neoadjuvant chemotherapy for the locally advanced gastric cancer according to claim 1, wherein the step of performing the data labeling and the data enhancement comprises:
labeling training data: labeling a tissue according to whether the tissue is a tumor tissue or not, and setting a discriminant threshold A here; when a proportion of fibrosis in a gray binary image and a gradient binary image of a block region after block processing exceeds the discriminant threshold A, the block region is considered to be the tumor tissue, and a label is set to 1; otherwise, the label is a fibrosis tissue, and set to be 0; and
enhancing the training data: the data enhancement comprises a rotation, a mirror image, and a gray transformation: the rotation is to rotate the block region according to a predetermined angle step; the mirror image is to turn the block region horizontally and vertically to obtain an image after a mirror operation; the gray transformation is to compress and stretch gray of the block region to obtain a plurality of groups of transformed pictures as the label set, and a transformation scale factor is δ.

6. The method for predicting the efficacy of the neoadjuvant chemotherapy for the locally advanced gastric cancer according to claim 1, wherein the step of establishing the deep perception network comprises:
S1: constructing an exponential linear unit (ELU) activation function to alleviate a disappearance of linear gradient;
S2: constructing a deep separable convolution module further by using the ELU activation function, wherein the deep separable convolution module comprises a 3×3 convolution and a 1×1 convolution, and an amount of convolution kernels is 64 and 128 respectively; and
S3: constructing a residual module by using the deep separable convolution module, wherein a combination of a residual module structure is as follows: the residual module structure comprises an uplink branch and a downlink branch, the uplink branch comprises a mean pooling layer, and the downlink branch comprises a 3×3 convolution layer→a first ELU activation layer→a deep separable convolution layer→a second ELU activation layer→an 1×1 convolution layer→a third ELU activation layer, wherein convolution kernel dimensions of the 3×3 convolution layer and the 1×1 convolution layer are 64 and 128 respectively; the uplink branch and the downlink branch are connected by a Contact layer finally.

7. The method for predicting the efficacy of the neoadjuvant chemotherapy for the locally advanced gastric cancer according to claim 6, wherein a structure of the deep perception network is: a data input layer→a first convolution layer→a second convolution layer→a first residual module→a second residual module→a third residual module→a third convolution layer→a global mean pooling→a softmax layer; wherein the first convolution layer, the second convolution layer, and the third convolution layer are all 3×3 convolution kernels, and kernel dimensions are 16, 32, and 128 respectively; the softmax layer outputs a final image block probability attribute, wherein a category corresponding to a high probability value is a category of an image block, and a calculation formula is:
$${y}_{i} = {S ( z )}_{i} = \frac{{e}^{{z}_{i}}}{{\sum }_{j = 1}^{C} {e}^{{z}_{j}}} , i = 1 , … , C ;$$
wherein z is an output of a previous layer, an input dimension of the softmax layer is C, and yᵢ is a probability that a predicted object belongs to class C.

8. The method for predicting the efficacy of the neoadjuvant chemotherapy for the locally advanced gastric cancer according to claim 1, wherein a constructing of the CNN-LSTM prediction model comprises: an input layer, a hidden layer, and an output layer; firstly, data is organized into a form recognized by the CNN-LSTM prediction model and imported into the CNN-LSTM prediction model through the input layer; then the data is processed through the hidden layer of a core part, wherein a CNN layer analyzes correlation features between the efficacy levels and the influencing factors, and then an LSTM layer extracts features of time series data in a time dimension; then, a complexity of the CNN-LSTM prediction model is increased through a Dense layer, and the data is mapped from a high dimension to a low dimension to retain useful information; meanwhile, a Dropout layer is connected after each layer to enhance robustness of the CNN-LSTM prediction model and prevent the CNN-LSTM prediction model from overfitting; finally, a predicted value is output through the output layer; an input of the CNN-LSTM prediction model is a two-dimensional matrix comprising the efficacy levels vₜ [symbol] vₜ₋₁ [symbol] . . . [symbol] v_{t-λ+1} and the influencing factors
$${f}_{t}^{( i )} , {f}_{t - 1}^{( i )} , … , {f}_{t - λ + 1}^{( i )} , ( i = 1 , 2 , 3 , … k ) ,$$
and a size of the two-dimensional matrix is (λ, k+1).

9. A system for predicting an efficacy of neoadjuvant chemotherapy for locally advanced gastric cancer, wherein the system is applied to the method for predicting the efficacy of the neoadjuvant chemotherapy for the locally advanced gastric cancer according to claim 1, comprising:
a data acquisition module, configured to acquire the historical image data of the local progression acquired through the multi-b-values non-Gaussian diffusion MRI and the corresponding prognostic image data acquired periodically, and divide the historical image data according to the preset b-value sequence to obtain the signal intensity data corresponding to each b-value;
a factor acquisition module, configured to acquire the voxel-related parameter and the diffusion-related parameter according to the signal intensity data combined with the intravoxel incoherent motion model and the diffusion kurtosis model; take the voxel-related parameter and the diffusion-related parameter as the influencing factors;
a label set establishment module, configured to select the optimal gray threshold for the segmentation of the prognostic image data, extract the interest region according to the optimal gray threshold; perform the gray segmentation and the gradient segmentation on the interest region respectively, and further adopt the two-peak iterative algorithm for the binary segmentation to obtain the gray binary image and the gradient binary image; merge to form the segmentation candidate region, divide the segmentation candidate region into the blocks, and further perform the data labeling and the data enhancement to obtain the label set;
a level division module, configured to establish the deep perception network, take the prognostic image data as the training set; input the label set and the training set into the deep perception network to obtain the tumor region and the fibrosis region to divide the efficacy levels; and
a level prediction module, configured to establish the data set by preprocessing the influencing factors and the efficacy levels; construct the CNN-LSTM prediction model, and set the initial parameter value of the CNN-LSTM prediction model simultaneously; adopt the data set to perform the Hyperparameter optimization to the CNN-LSTM prediction model to obtain the optimal model, and evaluate the chemotherapy efficacy level of the predicted image by the optimal model.

10. The system according to claim 9, wherein in the method, the preset b-value sequence comprises 0, 10, 20, 50, 100, 200, 400, 600, 800, 1000, 1500, and 2000; a unit is s/mm².

11. The system according to claim 9, wherein in the method, the voxel-related parameter comprises a slow diffusion coefficient, a fast diffusion coefficient, and a perfusion fraction; the diffusion-related parameter comprises an average diffusion rate and average diffusion kurtosis.

12. The system according to claim 9, wherein in the method, the optimal gray threshold comprises:
$${k}^{*} = {arg}_{k} ( {max}_{1 \leq k \leq L} {σ}_{B}^{2} ( k ) ) ;$$
wherein L is a level of a gray histogram; k is a gray threshold to distinguish a regional target from targets, the targets are significantly different from the regional targets;
$${σ}_{B}^{2}$$
is an overall inter-class difference of MRI images,
$${σ}_{B}^{2} = {w}_{1} {{w}_{2} ( {μ}_{1} - {μ}_{2} )}^{2} ,$$
w₁ is an appearing probability of the regional targets, w₂ is an appearing probability of the targets, μ₁ is an average gray of the regional targets, and μ₂ is an average gray of the targets.

13. The system according to claim 9, wherein in the method, the step of performing the data labeling and the data enhancement comprises:
labeling training data: labeling a tissue according to whether the tissue is a tumor tissue or not, and setting a discriminant threshold A here; when a proportion of fibrosis in a gray binary image and a gradient binary image of a block region after block processing exceeds the discriminant threshold A, the block region is considered to be the tumor tissue, and a label is set to 1; otherwise, the label is a fibrosis tissue, and set to be 0; and
enhancing the training data: the data enhancement comprises a rotation, a mirror image, and a gray transformation: the rotation is to rotate the block region according to a predetermined angle step; the mirror image is to turn the block region horizontally and vertically to obtain an image after a mirror operation; the gray transformation is to compress and stretch gray of the block region to obtain a plurality of groups of transformed pictures as the label set, and a transformation scale factor is δ.

14. The system according to claim 9, wherein in the method, the step of establishing the deep perception network comprises:
S1: constructing an ELU activation function to alleviate a disappearance of linear gradient;
S2: constructing a deep separable convolution module further by using the ELU activation function, wherein the deep separable convolution module comprises a 3×3 convolution and a 1×1 convolution, and an amount of convolution kernels is 64 and 128 respectively; and
S3: constructing a residual module by using the deep separable convolution module, wherein a combination of a residual module structure is as follows: the residual module structure comprises an uplink branch and a downlink branch, the uplink branch comprises a mean pooling layer, and the downlink branch comprises a 3×3 convolution layer→a first ELU activation layer→a deep separable convolution layer→a second ELU activation layer→an 1×1 convolution layer→a third ELU activation layer, wherein convolution kernel dimensions of the 3×3 convolution layer and the 1×1 convolution layer are 64 and 128 respectively; the uplink branch and the downlink branch are connected by a Contact layer finally.

15. The system according to claim 14, wherein in the method, a structure of the deep perception network is: a data input layer→a first convolution layer→a second convolution layer→a first residual module→a second residual module→a third residual module→a third convolution layer→a global mean pooling→a softmax layer; wherein the first convolution layer, the second convolution layer, and the third convolution layer are all 3×3 convolution kernels, and kernel dimensions are 16, 32, and 128 respectively; the softmax layer outputs a final image block probability attribute, wherein a category corresponding to a high probability value is a category of an image block, and a calculation formula is:
$${y}_{i} = {S ( z )}_{i} = \frac{{e}^{{z}_{i}}}{{\sum }_{j = 1}^{C} {e}^{{z}_{j}}} , i = 1 , … , C ;$$
wherein z is an output of a previous layer, an input dimension of the softmax layer is C, and yᵢ is a probability that a predicted object belongs to class C.

16. The system according to claim 9, wherein in the method, a constructing of the CNN-LSTM prediction model comprises: an input layer, a hidden layer, and an output layer; firstly, data is organized into a form recognized by the CNN-LSTM prediction model and imported into the CNN-LSTM prediction model through the input layer; then the data is processed through the hidden layer of a core part, wherein a CNN layer analyzes correlation features between the efficacy levels and the influencing factors, and then an LSTM layer extracts features of time series data in a time dimension; then, a complexity of the CNN-LSTM prediction model is increased through a Dense layer, and the data is mapped from a high dimension to a low dimension to retain useful information; meanwhile, a Dropout layer is connected after each layer to enhance robustness of the CNN-LSTM prediction model and prevent the CNN-LSTM prediction model from overfitting; finally, a predicted value is output through the output layer; an input of the CNN-LSTM prediction model is a two-dimensional matrix comprising the efficacy levels vₜ [symbol] vₜ₋₁ [symbol] . . . [symbol] v_{t-λ+1} and the influencing factors
$${f}_{t}^{( i )} , {f}_{t - 1}^{( i )} , … , {f}_{t - λ + 1}^{( i )} , ( i = 1 , 2 , 3 , … k ) ,$$
and a size of the two-dimensional matrix is (λ, k+1).
